(19) United States
(12) Patent Application Publication
(10) Pub. No.: US 2024/0099761 A1
(43) Pub. Date: Mar. 28, 2024

(54) SURGICAL DEVICE CONNECTOR

(71) Applicant: GYRUS MEDICAL LIMITED, Cardiff (GB)

(72) Inventor: David MORRIS, Cardiff (GB)

(73) Assignee: GYRUS MEDICAL LIMITED, Cardiff (GB)

(21) Appl. No.: 18/370,178

(22) Filed: Sep. 19, 2023

Related U.S. Application Data
(60) Provisional application No. 63/409,013, filed on Sep. 22, 2022.

(51) Int. Cl. A61B 18/12; A61B 90/00

(52) U.S. Cl. CPC A61B 18/12; A61B 90/08; A61B 2018/00178

(57) ABSTRACT

A surgical device includes a handpiece having a socket, including a distal opening providing access to a passage having a longitudinal axis, and first and second electrical contacts located therein, wherein the contacts are displaced along the longitudinal axis so the first contact is more distal than the second. Also, a surgical instrument includes an elongate shaft having distal and proximal ends, wherein an end effector at the distal end is for treating tissue. Further, a connecting portion includes third and fourth electrical contacts, wherein the contacts are displaced along the shaft so the third contact is more distal than the fourth. The surgical instrument is coaxially coupled to the handpiece by the connecting portion received in the passage, so the first contact is aligned with the third and the second is aligned with the fourth to provide an electrical power signal from the handpiece to the surgical instrument.

### TECHNICAL FIELD

The present disclosure relates to a surgical instrument for releasably coupling to a handpiece of a surgical device, and a handpiece for releasably coupling to a surgical instrument. The present disclosure also relates to a surgical device including a handpiece and a surgical instrument, a surgical apparatus, and a surgical system.

### BACKGROUND

Surgical (e.g. electrosurgical) devices provide advantages over traditional surgical devices in that they can be used for powered surgical purposes (e.g. ablation, coagulation and tissue sealing purposes). A powered surgical device may include a handle or handpiece coupled with a surgical instrument. The handle may receive an electrical power signal from a generator. The electrical power signal may then be transferred from the handle to the surgical instrument via electrical contacts, so that the instrument may perform surgical functions (e.g. tissue ablation and/or coagulation).

### SUMMARY OF THE DISCLOSURE

The present disclosure relates to a handpiece and a surgical instrument of a surgical device. The handpiece has a socket and electrical contacts located in the socket. The electrical contacts are displaced along a longitudinal axis of the socket. The surgical instrument includes a connecting portion also with electrical contacts. The electrical contacts of the surgical instrument are also displaced along a longitudinal axis of the instrument. The socket and the connecting portion of the handpiece are configured to be (e.g. coaxially) coupled to one another, by inserting the connecting portion into the socket. The electrical contacts of the instrument align and make connection with the electrical contacts in the socket, thereby enabling the transfer of electrical power between the handpiece and the instrument.

According to an aspect of the present disclosure, there is provided a surgical instrument for releasably coupling to a handpiece of a surgical device, the surgical instrument comprising:
an elongate shaft having a distal end and a proximal end, wherein the elongate shaft defines a longitudinal axis in a direction of elongation of the elongate shaft;
an end effector at the distal end of the elongate shaft, wherein the end effector is for treating tissue; and
a connecting portion at the proximal end of the elongate shaft, the connecting portion configured to be (e.g. coaxially) inserted into a socket of the handpiece and receive an electrical power signal from the handpiece, the connecting portion comprising:
   a first electrical contact configured to align with a corresponding first electrical contact in the socket of the handpiece when the connecting portion is in the socket, and
   a second electrical contact configured to align with a corresponding second electrical contact in the socket of the handpiece when the connecting portion is in the socket,
   wherein the first electrical contact and the second electrical contact are located at different longitudinal positions such that the first electrical contact is more distal than the second electrical contact.

In a second aspect of the present disclosure, there is provided a handpiece for releasably coupling with a surgical device, the handpiece comprising:
a socket for (e.g. coaxially) receiving a connecting portion of the surgical instrument to provide an electrical power signal to the surgical instrument, the socket comprising:
a distal opening that provides access to a passage defined by an inner wall of the socket, the passage having a longitudinal axis;
   a first electrical contact located in the passage and configured to align with a corresponding first electrical contact of the connecting portion of the surgical instrument when the connecting portion is inserted in the socket, and
a second electrical contact located in the passage and configured to align with a corresponding second electrical contact of the connecting portion of the surgical instrument when the connecting portion is inserted in the socket,
wherein the first electrical contact and the second electrical contact are located at different longitudinal positions such that the first electrical contact is more distal than the second electrical contact.

In a third aspect of the present disclosure, there is provided a surgical apparatus (e.g. a kit) comprising the surgical instrument of the first aspect, and the handpiece of the second aspect.

In a fourth aspect of the present disclosure, there is provided a surgical device comprising the surgical instrument of the first aspect, and the handpiece of the second aspect, wherein the connecting portion of the surgical instrument is (e.g. coaxially) received in the socket of the handpiece.

In a fifth aspect of the present disclosure there is provided a surgical device comprising:
a handpiece having a socket comprising:
   a distal opening that provides access to a passage defined by an inner wall of the socket, the passage having a longitudinal axis, and a first electrical contact and a second electrical contacted each located in the passage, wherein the first electrical contact and the second electrical contact are displaced along the longitudinal axis of the passage such that the first electrical contact is more distal than the second electrical contact; and
      a surgical instrument comprising:
an elongate shaft having a distal end and a proximal end, wherein the elongate shaft defines a longitudinal axis in a direction of elongation of the elongate shaft,
an end effector at the distal end of the elongate shaft, wherein the end effector is for treating tissue, and
   a connecting portion comprising a third electrical contact and a fourth electrical contact, wherein the third electrical contact and the fourth electrical contact are displaced along the longitudinal axis of the elongate shaft such that the third electrical contact is more distal than the fourth electrical contact,
wherein the surgical instrument is (e.g. coaxially) coupled to the handpiece by the connecting portion being received in the passage, such that the first electrical contact is aligned with the third electrical contact and the second electrical contact is aligned with the fourth electrical contact to provide an electrical power signal from the handpiece to the surgical instrument.

In a sixth aspect of the present disclosure, there is provided a surgical system, comprising:
an electrical generator;
a suction source; and
a surgical device according to any of fourth or fifth aspects, the arrangement being such that in use the generator supplies an electrical power signal to the handpiece

Other features of the disclosure are described below and recited in the appended claims.

### BRIEF DESCRIPTION OF THE DRAWINGS

Examples of the present disclosure are now described with reference to the accompanying drawings, in which:

FIG. 1 shows an example of the electrosurgical instrument system including an RF electrosurgical device, according to an example of the present disclosure;

FIG. 2A shows a perspective view of a portion of a handpiece of an electrosurgical device, according to an example of the present disclosure;

FIG. 2B shows a side cross-sectional view of the handpiece portion of FIG. 2A;

FIG. 3A shows a perspective view of a proximal end of an electrosurgical instrument of an electrosurgical device, according to an example of the present disclosure;

FIG. 3B shows a side view of the proximal end of the electrosurgical instrument shown in FIG. 3A;

FIG. 3C shows a magnified view of the electrosurgical instrument of FIGS. 3A and 3B;

FIG. 4 shows a side cross-sectional view of an electrosurgical device including the handpiece portion of FIGS. 2A-2B and the electrosurgical instrument of FIGS. 3A-3C;

FIG. 5 shows an electrical contact according to an example of the present disclosure; and

FIG. 6 shows a sealing element according to an example of the present disclosure.

### DETAILED DESCRIPTION

The present disclosure relates to a surgical device including a handle and a detachable/releasable powered surgical instrument. The handle includes a socket with active and return electrical contacts inside the socket. The instrument includes a connecting portion for inserting into the socket. The connecting portion also includes active and return electrical contacts. When the connecting portion is inserted into the socket, the active contact and the return contact of the instrument align and connect with the active contact and the return contact in the socket, respectively. Electrical power may therefore be provided from the handle to the instrument, via the electrical contacts.

The active contacts and the return contacts are usually in relatively close proximity to one another. As such, there is a risk that a short circuit (e.g. spark gap) may form directly between the active and return contacts. Therefore, it can be desirable to electrically isolate the active contacts from the return contacts. Accordingly, the active and return contacts of the instrument may be displaced along a length of the instrument. The active and return contacts in the socket may also be displaced along a length of the socket. When the connecting portion of the instrument is inserted into the socket, the active and return contacts of the instrument may align and connect with the active and return contacts of the socket, respectively. The connecting portion of the instrument may also include a sealing element positioned in between the active and return contacts of the instrument. When the connecting portion of the instrument is inserted in the socket, the sealing element may electrically isolate both active contacts from both return contacts whilst the instrument is in the socket.

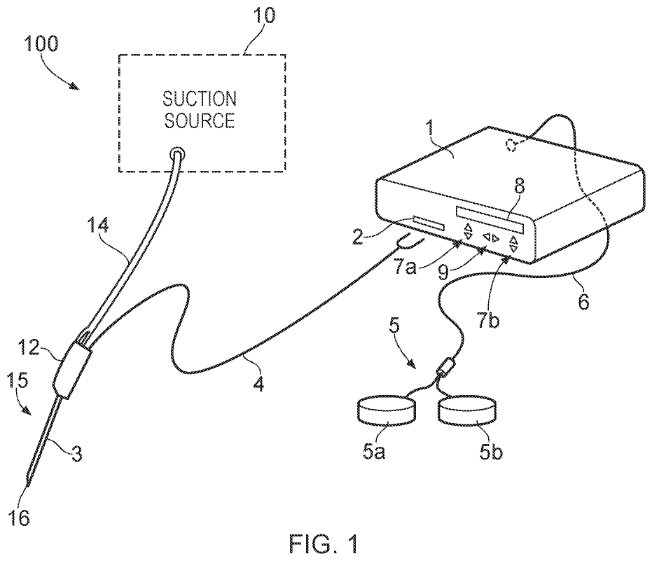

FIG. 1 shows an electrosurgical system 100 including an electrosurgical generator 1 and an electrosurgical device 15. The electrosurgical generator 1 is configured to generate an electrical radio frequency (RF) power signal. The electrosurgical generator 1 has an output socket 2 that provides the electrical RF power signal, via a connection cord 4, to the electrosurgical device 15.

The electrosurgical device 15 includes a handpiece 12 and an electrosurgical instrument 3. The handpiece 12 is detachably or releasably coupled to the electrosurgical instrument 3. As described below, the handpiece 12 includes a socket for receiving a proximal end of the electrosurgical instrument 3, to detachably couple the electrosurgical instrument 3 to the handpiece 12. The electrosurgical instrument 3 includes an end effector 16 at a distal end thereof. The handpiece 12 is configured to receive the electrical RF power signal via the connection cord 4. Alternatively, the handpiece 12 may generate the electrical RF power signal, e.g. using one or more batteries. The electrosurgical device is configured to provide the electrical RF power signal from the handpiece 12 to the electrosurgical instrument 3. The electrosurgical instrument 3 is configured to conduct or otherwise provide the electrical RF power signal to the end effector 16.

As described in more detail below, the handpiece 12 includes an active electrical contact and a return electrical contact (not shown). The electrosurgical instrument 3 also includes an active electrical contact and a return electrical contact (not shown). The active and return electrical contacts are arranged such that the active contact of the handpiece 12 aligns with and makes electrical contact with the active contact of the electrosurgical instrument 3 when the handpiece 12 and the electrosurgical instrument 3 are coupled (i.e. when the electrosurgical instrument 3 is received in a socket of the handpiece 3). Furthermore, the return contact of the handpiece 12 aligns with and makes electrical contact with the return contact of the electrosurgical instrument 3 when the handpiece 12 and the electrosurgical instrument 3 are coupled. As such, the active contacts and the return contacts provide an electrical connection between the handpiece 12 and the electrosurgical instrument 3 when they are coupled to one another. The electrical RF power signal is delivered from the handpiece 12 to the electrosurgical instrument 3 via the active contacts and the return contacts, i.e. via the electrical connection provided by the contacts. The electrical RF power signal is then received by the end effector 16 from the active and return contacts of the electrosurgical instrument 3. The electrical RF power signal may be delivered to the end effector 16 from the active and return contacts of the electrosurgical instrument 3 via respective active and return current paths.

The end effector 16 is configured to deliver the electrical RF power signal to a surgical site in order to treat tissue. For example, the end effector 16 may be configured to perform ablation (e.g. cutting or vaporisation) or coagulation functionalities at the surgical site using the electrical RF power signal. The end effector 16 may be any type of end effector known in the art. For example, the end effector 16 can include an active electrode and a return electrode which receive the electrical RF power signal via active and return current paths. The active and return electrodes may deliver the RF power signal to the surgical site to perform electrosurgical functions, such as tissue ablation and/or coagulation.

In some examples, the electrosurgical device 15 is also configured to perform mechanical shaving/cutting of tissue. For example, the electrosurgical instrument 3 can include a rotatable shaft with a cutting blade at a distal end of the rotatable shaft (not shown). The handpiece 12 can include drive componentry (e.g. a motor) to rotate the rotatable shaft to perform mechanical cutting/shaving at the surgical site (not shown). The drive componentry can be powered by the generator 1 via the cord 4. The electrosurgical device 15 may be configured to simultaneously perform mechanical shaving and ablation/coagulation.

Activation of the generator 1 may be performed at the device 15 via a hand switch (not shown) on the handpiece 12 of the device 15, or by means of a footswitch unit 5 connected separately to the rear of the generator 1 by a footswitch connection cord 6. In the illustrated example, the footswitch unit 5 has two footswitches 5a and 5b for selecting a coagulation mode, or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes. If the electrosurgical device 15 also performs mechanical cutting, then additional switching means similar to those described above may be provided for selecting a mechanical shaving mode.

The system 100 may optionally include a suction source 10. The electrosurgical device may be connected to the suction source 10 via a suction tube 14. The instrument 3 may include a suction aperture which is operatively connected to the suction source via the suction tube 14, in order to clear the surgical site of debris.

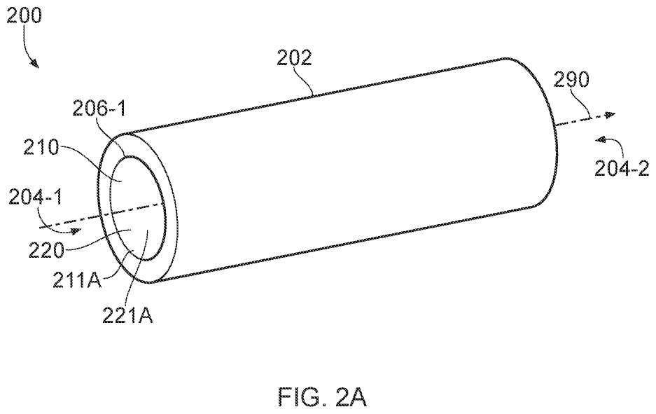

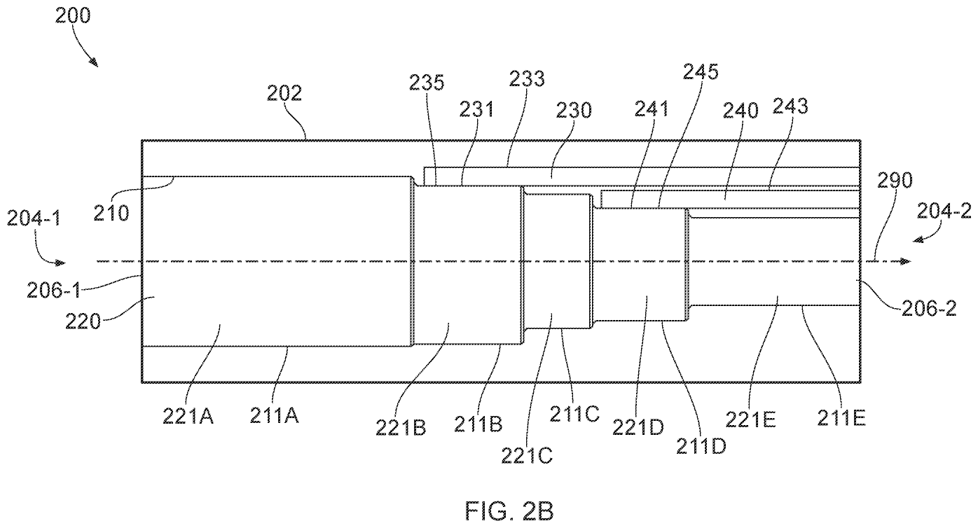

FIGS. 2A and 2B show a socket portion 200 of the handpiece 3 according to an example of the present disclosure. The socket portion 200 enables the electrosurgical instrument 15 to be detachably coupled to the handpiece 3. As described below, the socket portion 200 provides a passage or socket configured to receive a proximal end of the electrosurgical instrument 15. Moreover, the socket portion 200 includes electrical contacts which will align with electrical contacts of the electrosurgical instrument 15 when the proximal end of the electrosurgical instrument 15 is received in the socket portion 200, to enable the electrical RF power signal to be provided from the handpiece 3 to the electrosurgical instrument 15.

The socket portion 200 has a body 202. In the present example, the body 202 is substantially cylindrical. The body 202 has a distal side 204-1 and a proximal side 204-2. The distal side 204-1 has a distal opening 206-1 and the proximal side 204-2 has an optional proximal opening 206-2. The distal opening 206-1 and optionally the proximal opening 206-2 provide access to a passage 220 defined by an interior wall 210 of the body 202. The passage 220 extends between the distal opening 206-1 and the proximal opening 206-2 and has a longitudinal axis 290. The passage 220 is coaxial with the openings 206-1 and 206-2. The socket portion 200 is configured to couple with the electrosurgical instrument 3 by receiving a proximal end of the electrosurgical instrument 3 in the passage 200, via the distal opening 206-1. The passage 220 may be considered as a socket. The body 202 is electrically non-conductive. The body 202 may be made of e.g. a plastic, or a ceramic.

The passage 220 has a length in a longitudinal direction of the socket portion (e.g. as indicated by the direction of the axis 290). The passage 220 also has a radius in a direction perpendicular to the axis 290. The radius of the passage 220 decreases in the distal-proximal direction (i.e. in the direction from the distal end 204-1 to the proximal end 204-2). In particular, the passage 220 includes a series of coaxial sections or segments 221A, 221B, 221C, 221D, 221E in between the distal opening 206-1 and the proximal opening 206-2. The passage section 221E may be optional. Each of the passage sections 221A-221E is coaxial with the axis 290. The passage sections 221A, 221B, 221C, 221D, 221D, 221E are defined by respective sections 211A, 211B, 211C, 211D, 211D, 211E of the interior wall 210. Each of the passage sections 221A-221E have a substantially circular cross section. As such, each passage section 221A-221E may be considered as having or occupying a substantially cylindrical shape or volume. Each passage section 221A-221E has a length in a longitudinal direction of the socket portion 200 (e.g. as indicated by the direction of the axis 290). Each passage section 221A-221E also has a radius. Each radius is in a direction perpendicular to the axis 290 (i.e. each radius corresponds to the radius of the cross-section of the respective passage section). The radius of each passage section 221A-221E may be substantially uniform across the length of the respective passage section. The passage sections 221A-221E have different radiuses. In particular, the radiuses of the passage sections 221A-221E decrease in the distal-proximal direction (i.e. in the direction from the distal end 204-1 to the proximal end 204-2). More particularly, the distal-most section 221A has the largest radius. Moving in the proximal direction, the section 221B has a smaller radius than the section 221A. The section 221C has a smaller radius than the section 221B. The section 221D has a smaller radius than the section 221C. The section 221E has a smaller radius than the section 221D. As such, the total radius of the passage 220 decreases in the distal-proximal direction, e.g. in a stepwise manner. In some examples, the lengths of each section 221A-221E may vary. In the illustrated example, the passage section 221A may have the longest length. The passage section 221B may have a shorter length than the passage section 221A. The passage section 221C may have a shorter length than the passage section 221B. The passage section 221D may have a length that is in between the lengths of the passage sections 221B and 221C. The passage section 211E may have a length that is longer than the section 221B, optionally also longer than the section 221A.

The body 202 further includes a cavity 233. The wall section 211B also includes an aperture 235 which provides access to the cavity 233. The socket portion 200 includes an active plate 230 which is held within the cavity 233. The active plate 230 is electrically conductive, and is for conducting an electrical signal (e.g. an electrical RF power signal). Because of the aperture 235, a portion 231 of the active plate is exposed to the passage 220. In particular, the portion 231 is exposed to the passage section 221B. The portion 231 may be considered as forming part of the wall section 211B. The portion 231 may be considered as being embedded in the wall section 211B. The portion 231 may be substantially flat and/or flush with the wall section 211B. The portion 231 of the active plate 230 may be considered as an active electrical contact. The body 202 also includes a cavity 243. The wall section 211D also includes an aperture 245 which provides access to the cavity 243. The socket portion 200 further includes a return plate 240 which is held within the cavity 243. The return plate 240 is electrically conductive and is for conducting an electrical signal (e.g. an electrical RF power signal). Because of the aperture 245, a portion 241 of the active plate is exposed to the passage 220. In particular, the portion 241 is exposed to the passage section 221D. The portion 241 may be considered as forming part of the wall section 211D. The portion 241 may be considered as being embedded in the wall section 211D. The portion 241 may be substantially flat and/or flush with the wall section 211D. The portion 241 may be considered as a return electrical contact.

As such, the active electrical contact 231 and the return electrical contact 241 are displaced along the longitudinal axis 290 (i.e. located at different longitudinal positions along the length of the passage 220, at different positions along the axis 290). In particular, the active electrical contact 231 is more distal than the return electrical contact 241. Furthermore, the active electrical contact 231 and the return electrical contact 241 are located at different distances from the axis 290 (i.e. at different radiuses relative to the axis 290). In particular, the active electrical contact 231 is further from the axis 290 than the return electrical contact 241.

As described further below, when the electrosurgical instrument 3 is received in the passage 220, the active electrical contact 231 may align with and make electrical connection with an active electrical contact of the electrosurgical instrument 3. The return electrical contact 241 may align with and make electrical connection with a return electrical contact of the electrosurgical instrument 3. As such, an electrical connection is made between the handpiece 12 and the electrosurgical instrument 3. This may allow the electrical RF power signal to be provided from the handpiece 12 to the electrosurgical instrument 3. Furthermore, the electrosurgical instrument 3 may include a sealing element that engages the wall section 211C when the electrosurgical instrument 3 is received in the passage, to electrically isolate the active and return contacts from one another. The electrosurgical instrument 3 may include a sealing element that engages the wall section 211A when the electrosurgical instrument 3 is received in the passage, to electrically isolate the active contact from the external environment (e.g. from the distal opening 206-1). Although not shown, it will be appreciated that the active plate 230 and the return plate 240 may be connected to the cord 4 to receive the RF power signal from the generator 1 (e.g. via electrical connections at the proximal side 204-2 of the cavities 233 and 243), or connected to a power supply (e.g. one or more batteries) to self-generate the RF power signal.

It will be appreciated that the Figures show a simplified view of the socket portion 200 of the handpiece 12. For example, it will be appreciated that the body 202 of the socket portion 200 may not necessarily be cylindrical. Rather the body 202 may be any suitable shape in which the passage 220 can be formed.

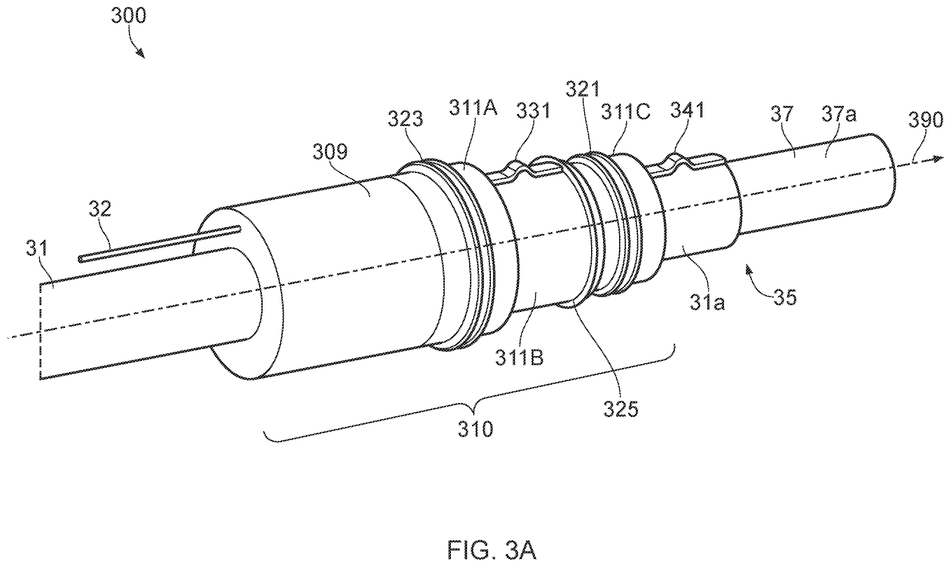

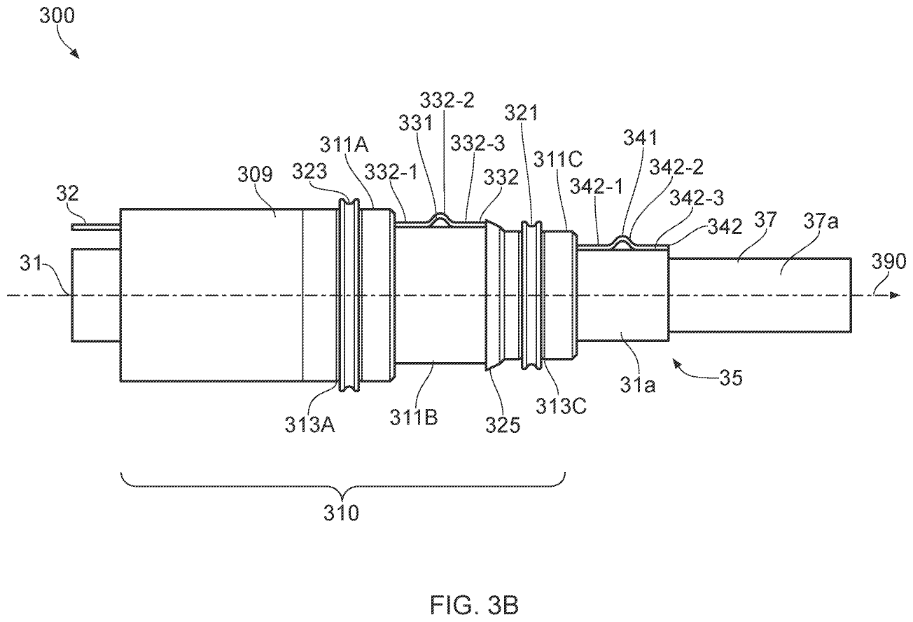

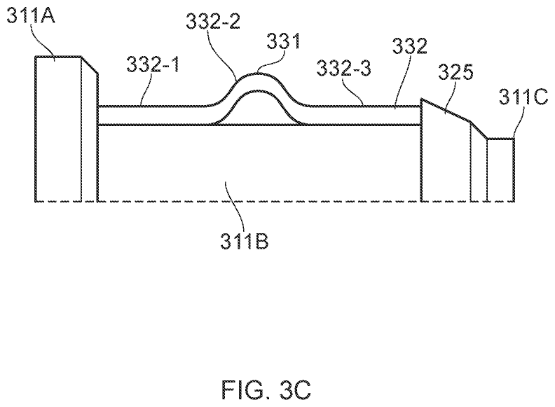

FIGS. 3A and 3B show a proximal end portion 300 of the electrosurgical instrument 3 according to an example of the present disclosure. FIG. 3C shows a magnified region of the proximal end portion 300. The proximal end portion 300 may be considered as a connecting portion of the electrosurgical instrument 3. As described below, the connecting portion 300 enables the electrosurgical instrument 3 to be detachably coupled to the handpiece 12. In particular, the connecting portion 300 is configured to be received in the passage 220 of the socket portion 200 of the handpiece 12, via the distal opening 206-1 of the socket portion 200. The connecting portion 300 includes electrical contacts that will align and make electrical connection with the electrical contacts 231 and 241 inside the socket portion 200, thereby enabling transfer of the electrical RF power signal from the handpiece 12 to the electrosurgical instrument 3.

The electrosurgical instrument 3 has an elongate shaft 31 having a proximal end 35, a distal end (not shown in FIGS. 3A-3B) and an axis 390. The end effector 16 is located at the distal end of the elongate shaft 31 (not shown). The connecting portion 300 is at the proximal end 35 of the elongate shaft 31. The connecting portion 300 includes a connection hub 310 having a body 309 that is coaxial with the axis 390. The proximal end 35 of the elongate shaft 31 extends through the body 309, such that a proximal portion 31a of the elongate shaft 31 protrudes from a proximal side of the body 309. The proximal portion 31a of the elongate shaft 31 may be considered as part of the connecting portion 300. The body 309 is coaxial with the elongate shaft 31 about the axis 390. Optionally, the electrosurgical instrument 3 includes a rotatable shaft 37. The rotatable shaft 37 is for providing mechanical shaving and/or cutting functionalities. For example, the rotatable shaft may have a cutting blade at its distal end (not shown). The rotatable shaft is positioned concentrically within the elongate shaft 31, such that the rotatable shaft 37 is coaxial with the axis 390. A proximal portion 37a of the rotatable shaft 37 protrudes from the proximal end 35 of the elongate shaft 31. The rotatable shaft 37 is rotatable within the elongate shaft 31 whilst the elongate shaft 31 remains stationary or fixed.

The hub body 309 has a length in a longitudinal direction of the elongate shaft 31 (e.g. as indicated by the direction of the axis 390). The hub body 309 also has a radius in a direction perpendicular to the axis 390. Similarly to the passage 220, the radius of the body 309 decreases in the distal-proximal direction (i.e. in the direction from the distal end of the elongate shaft 31 to the proximal end 35 of the elongate shaft 31). In particular, the body 309 includes a series of coaxial sections 311A, 311B, 311C. Each of the sections 311A-311C are coaxial with the axis 390. Each of the sections 311A-311C have a substantially circular cross section. As such, each body section 311A-311C may be considered as having a substantially cylindrical shape. Each body section 311A-311C has a length in the longitudinal direction of the elongate shaft 31. Each body section 311A-311C also has a radius. Each radius is in a direction perpendicular to the axis 390 (i.e. each radius corresponds to the radius of the cross-section of the respective body section). The radius of each body section 311A-311C may be substantially uniform across at least the majority of the length of the respective body section. However, the body sections 311A-311C have different radiuses. Like the passage sections 221A-221C, the radiuses of the body sections 311A-311C decrease in the distal-proximal direction (i.e. in the direction of the arrow 390). More particularly, the distal-most section 311A has the largest radius. Moving in the proximal direction, the body section 311B has a smaller radius than the section 311A. The section 311C has a smaller radius than the section 311B. As such, the total radius of the body 309 decreases in the distal-proximal direction, e.g. in a stepwise manner.

The proximal portion 31a of the elongate shaft 31 has a radius that is smaller than the radius of the body section 311C. The proximal portion 37a may have a radius that is smaller than the radius of the proximal portion 31a of the elongate shaft 31. As such, the total radius of the connecting portion decreases in the distal-proximal direction, e.g. in a stepwise manner.

The connection hub 310 includes an active electrical contact 331 and a return electrical contact 341. The active electrical contact 331 is positioned on a surface of the body section 311B. The return electrical contact 341 is positioned on a surface of the proximal portion 31a of the elongate shaft 31. As such, the active electrical contact 331 and the return electrical contact 341 are displaced along the longitudinal axis 390 (i.e. located at different longitudinal positions along the length of the electrosurgical instrument 3, along the axis 390). In particular, the active electrical contact 331 is positioned more distally than the return electrical contact along the length of the instrument 3 (e.g. along the axis 390). Moreover, since the body section 311B and the proximal portion 31a have different radiuses, their surfaces are at different distances from the axis 309. In particular, the surface of the body section 311B is at a greater distance from the axis 390. As such, the electrical contacts 331 and 341 are located at different distances from the axis 309 (i.e. radially from the axis 390). In particular, the active electrical contact 331 is at a greater distance from the axis 390 than the return electrical contact 341.

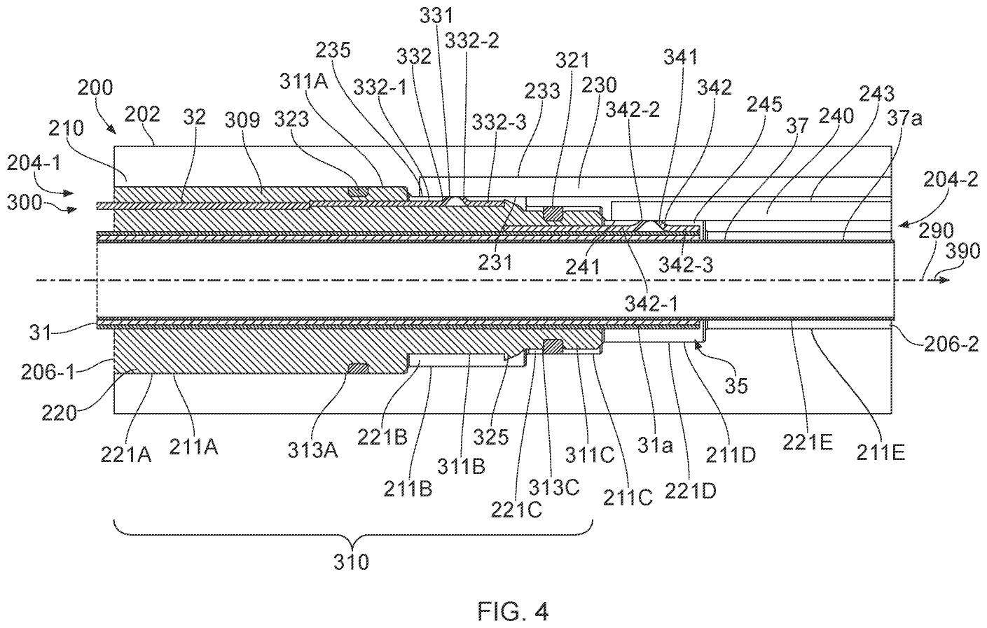

The active electrical contact 331 can be seen in more detail in FIG. 3C. The active electrical contact 331 is formed of a strip 332 of electrically conductive material. The material of the strip 332 is also resiliently deformable. As such, the active electrical contact 331 is both electrically conductive and resilient deformable. The active electrical contact 331 may be considered as a leaf spring. The active electrical contact 331 may be considered as a leaf spring. The strip 332 includes a first (e.g. distal) flat portion 332-1, a second (e.g. proximal) flat portion 332-3, and a crimped or raised portion 332-2 in between the flat portions 332-1 and 332-3. The flat portions 332-1 and 332-3 lie on a plane of the strip 332. The crimped portion 332-2 has a height, e.g. above the plane of the strip 332. The strip 332 is positioned on the surface of the body section 311B such that the strip 332 extends in the longitudinal direction of the elongate shaft 31. The crimped portion 332-2 therefore has a height above the surface of the body section 311B (e.g. in a radial direction from the axis 390). The active contact 331 may therefore be resilient deformable in a direction in a direction perpendicular to the axis 390, in particular towards the axis 390. Optionally, the flat portion 332-1 of the active contact 331 may extend into the body section 311A, e.g. to better secure the active contact on the surface of the body section 311B (e.g. see FIG. 4). In some examples, the body section 311B may include a lip or rim 325 around the periphery of its proximal edge. The flat portion 332-3 may abut the lip or rim 325. The lip or rim 325 may help to secure the active electrical contact 331 on the surface of the body section 311B.

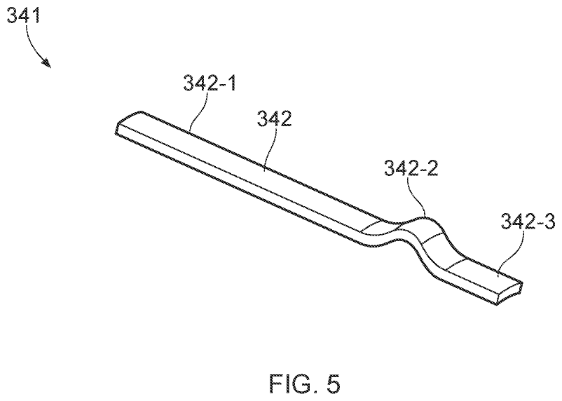

The return electrical contact 341 can be seen in more detail in FIG. 5. Like the active electrical contact 331, the return electrical contact 341 is formed of a strip 342 of electrically conductive material. The material of the strip 342 is also resiliently deformable. As such, the return electrical contact 341 is both electrically conductive and resilient deformable. The return electrical contact 341 may be considered as a leaf spring. The strip 342 includes a first (e.g. distal) flat portion 342-1, a second (e.g. proximal) flat portion 342-3, and a crimped or raised portion 342-2 in between the flat portions 342-1 and 342-3. The flat portions 342-1 and 342-3 lie on a plane of the strip 342. The crimped portion 342-2 has a height, e.g. above the plane of the strip 342. The strip 342 is positioned on the surface of the proximal portion 31a of the elongate shaft 31, such that the strip 342 extends in the longitudinal direction of the elongate shaft 31. The crimped portion 342-2 therefore has a height above the surface of the portion 31a of the elongate shaft 31. The return contact 341 may therefore be resilient deformable in a direction perpendicular to the axis 390, in particular towards the axis 390. Optionally, the flat portion 342-1 of the return contact 341 may extend into the body section 311C, e.g. to better secure the return contact on the surface of the elongate shaft 31 (e.g. see FIG. 4). It will be appreciated that the features of the return electrical contact 341 depicted and described in relation to FIG. 5 may also apply to the active electrical contact 331.

The electrosurgical instrument 3 includes a wire 32 which provides an active current path between the active electrical contact 331 and the end effector (not shown). For example, the wire 32 may provide a current path to an active electrode of the end effector. Although not shown in FIGS. 3A-3C, the flat portion 332-1 of the active contact 331 may extend into the body section 311A to connect to the wire 32 (e.g. see FIG. 4). Moreover, the elongate shaft 31 provides a return current path between the return electrical contact 341 and the end effector (not shown). For example, the elongate shaft may provide a current path from a return electrode of the end effector (not shown). The elongate shaft 31 is made from an electrically conductive material in order to provide the electrically conductive return path.

The connection hub 310 also includes sealing elements 321 and 323. In the present example, the sealing elements 321 and 323 are sealing rings. The sealing ring 321 is located around the surface of the body section 311C and is coaxial with the axis 390. In particular, the surface of the body section 311C includes a groove 313C that is coaxial with the axis 390, and the sealing ring 321 is positioned in the groove 313C. The sealing ring 323 is located around the surface of the body section 313A and is coaxial with the axis 390. In particular, the surface of the body section 311A includes a groove 313A that is coaxial with the axis 390, and the sealing ring 323 is positioned in the groove 313A. As such, the sealing rings 321 and 323 are located at different longitudinal positions along the length of the instrument 3. The sealing rings 321 and 323 are also coaxial with one another. Moreover, each of the sealing rings 321, 323, and the electrical contacts 331, 341, are at different positions along the length of the instrument 3. However, the sealing rings 321, 323 may have different radiuses or diameters. In particular, the sealing ring 323 may have a larger diameter in order to fit around the surface of the larger body section 311A (e.g. in the groove 313A). The sealing ring 321 may have a relatively smaller diameter in order to fit around the surface of the smaller body section 311C (e.g. in the groove 313C).

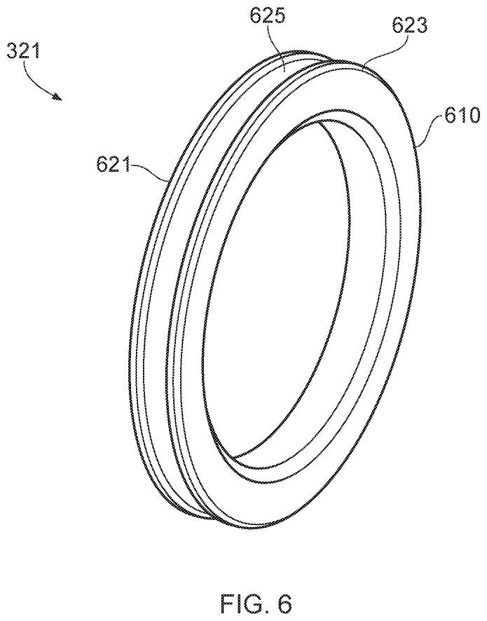

FIG. 6 shows the sealing element 321 in more detail. The sealing element 321 has a ring-shaped body 610 made of a resiliently deformable material. The material of the body 610 is also electrically insulating. As such, the sealing element 321 is both resiliently deformable and electrically insulating. The body 610 has a first lip 621 and a second lip 623, both of which run around the circumference of the body 610. The lips 621 and 623 are coaxial with one another and extend in a radial direction, to define a coaxial groove 625 in between the lips. The lips 621 and 623 may improve the sealing functionality of the sealing element 321. It will be appreciated that the features of the sealing element 321 depicted and described in relation to FIG. 6 may also apply to the sealing element 323.

FIG. 4 shows a side cross-sectional view of the connecting portion 300 coupled with the socket portion 200. In particular, the connecting portion 300 is inserted into the passage or socket 220, via the opening 206-1. In the coupled arrangement, the passage 220 and the connecting portion 300 share the same axis 290/390. Therefore, the connecting portion 300 is coaxially coupled with (e.g. received in) the socket 200, such that the axis 290 of the passage 220 and the axis 390 of the elongate shaft 31 align. As shown, the passage 220 and the connecting portion 300 are cooperatively dimensioned so that the connecting portion 300 can be received in the passage 200. In particular, sections 311A-311C, 31a, 37a of the connecting portion 300 are configured to be received in respective sections 221A-221E of the passage 200. As shown, the proximal portion 37a of the rotatable shaft 37 is received in the passage section 221E. The proximal portion 31a of the elongate shaft 31 is received in the passage section 221D. The sections of the hub body 309 are received in the passage sections 221A-221C. In particular the body section 311C is received in the passage section 221C. The body section 311B is received in the passage section 221B. The body section 311A is received in the passage section 221A. Although not shown, the proximal portion 37a of the rotatable shaft 37 may couple to drive componentry in the handle 12 to rotate the rotatable shaft 37 within the elongate shaft 31.

As shown in the coupled arrangement of FIG. 4, the active electrical contact 331 of the connecting portion 300 aligns with and makes electrical connection with the active electrical contact 231 of the socket portion 200. In particular, the crimped portion 332-2 of the active electrical contact 331 aligns with and makes contact with the active electrical contact 231 of the socket portion 200. The return electrical contact 341 of the connecting portion 300 aligns with and makes electrical connection with the return electrical contact 241 of the socket portion 200. In particular, the crimped portion 342-2 of the return electrical contact 341 aligns with and makes contact with the return electrical contact 241 of the socket portion 200. As such, when the handpiece 12 is coupled with the instrument 3, the electrical RF power signal may be provided to the instrument 3, via the pairs of active and electrical contacts. Since the electrical contacts 331 and 341 are resiliently deformable, the contacts 231 and 241 of the socket portion 200 may exert a force against the crimped portions 332-2 and 342-2 causing the electrical contacts 331 and 341 to deform and store elastic energy. The contacts 331 and 341 may therefore exert a force back against the respective contacts 231 and 241 of the socket portion 200. This may enable a more secure electrical connection between the pairs of contacts 231/331 and 241/341.

As also shown in FIG. 4, the sealing ring 321 abuts and/or sealingly engages the wall section 211C. The sealing ring 321 may engage the wall section 211C along an entire circumference of the wall section 211C. In particular, the lips of the sealing ring 321 may engage the wall section 211C. The sealing ring 321 may therefore electrically isolate the active contacts 331/231 from the return contacts 341/241. Furthermore, the sealing ring 323 abuts or engages the wall section 311A. The sealing ring 323 may engage the wall section 211A along an entire circumference of the wall section 211A. In particular, the lips of the sealing ring 323 may engage the wall section 211A. The sealing ring 323 may therefore isolate the active contacts 331/231 (and the return contacts 341/241) from the external environment (e.g. from the distal opening 206-1 to the passage 220). The sealing ring 323 may provide electrical isolation. The sealing ring 321 and/or 323 may provide a watertight seal.

Advantageously, the active contacts 231/331 and the return contacts 241/341 are displaced along the axis 290/390. This may enable the use of the sealing element 321 in between the active and return contacts, in order to seal the active contacts 231/331 off from the return contacts 241/341. More advantageously, a special seal design is not necessary (e.g. an off the shelf sealing ring can be used, such as an o-ring or any other similar sealing ring), which reduces the manufacturing cost of the electrosurgical device 15. The connecting portion 300 and the socket 220 can reduce in radius in the distal-proximal direction in a stepwise manner, which may improve the functionality of the seals whilst making it easier to insert the connection portion 300 into the socket 220. Additionally, in an example implementation, the handpiece 12 may be a reusable component of the device 15 and the instrument 3 may be disposable. The deformable elements of the electrosurgical device 15 (e.g. the seals and leaf spring contacts) may be more susceptible to wear and damage. Advantageously, the sealing elements 321/323 and the leaf spring contacts 331/341 are all provided on the disposable instrument 3. As such, the handpiece 12 may be easier to clean and maintain, and also be more robust against long term wear and damage.

It will be appreciated that the exact dimensions of the connecting portion 300, the passage 200, and the features thereof may vary depending on the implementation. Rather, any appropriate dimensions are within the scope of the present disclosure, provided that it enables the present invention to function as described herein. In particular, any appropriate dimensions are within the scope of the present disclosure that enables each section 311A-311C, 31a, 37a of the connecting portion 300 to be received in the respective passage section 221A-221E, and enables the electrical contacts and the seals to align and function as described herein. In some examples, the length of each section 311A-311C, 31a, 37a of the connecting portion 300 is substantially the same as the length of the respective passage section 221A-221E. The radius of each section 311A-311C, 31a, 37a of the connecting portion 300 is generally less than the radius of the respective passage section 221A-221E, to enable the section of the connecting portion 300 to be received in the respective passage section. The electrical contacts 331 and 341 have an appropriate height to engage the respective contacts 231, 241 when the connecting portion 300 is inserted into the passage 220. In particular, the crimped portions 332-2 and 342-2 may have an appropriate height to engage the respective contacts 231 and 241 when the connecting portion 300 is inserted into the passage 220. The seals 321 and 323 have an appropriate height and/or thickness to appropriately engage the respective walls 211C and 211A. In particular, the lips of the seals 321 and 323 may have an appropriate height and/or thickness to sealingly engage the respective wall sections 211C and 211A. In some examples, the radius of each section of the connecting portion 300 is dimensioned to prevent the section of the connecting portion 300 from entering the proximally adjacent passage section (e.g. by abutting the edge of the proximally adjacent passage section).

Although not shown, in some examples the socket portion 200 and the connecting portion 300 can include a cooperating locking mechanism. The locking mechanism may secure the coupling between the handle 12 and the instrument 3. In particular, the locking mechanism may lock the connecting portion 300 in the passage 220.

It is described above how the present invention enables a RF electrical power signal to be provided from the handle 12 to the instrument 3. However, it will be appreciated that the present invention is not limited to use with a RF power signal, and rather the present invention can be used with any type of electrical power signal. Moreover, the invention is not limited to use with an electrical power signal, and can be used to enable any other type of electrical signal to be provided from the handle 12 to the instrument 3. For example, the invention can be used to transfer an electrical signal that encodes information or data (e.g. a device signature or identification information) from the handle 12 to the instrument 3.

In some examples, the instrument 3 may not be an electrosurgical instrument. Rather, the techniques of the present disclosure can be used to deliver electrical power to any type of surgical instrument (e.g. powered surgical instrument) having any type of surgical end effector.

In some examples, the connecting portion 300 may include multiple active contacts on the surface of the body section 311B, e.g. at different circumferential positions about the axis 390. Alternatively or additionally, the connecting portion 300 may include multiple return contacts on the surface of the proximal portion 31a of the elongate shaft 31, e.g. at different circumferential positions about the axis 390. The active plate 230, the return plate 240, the aperture 235 and the aperture 245 may extend around the axis 290, to provide multiple contact points around the circumferences of the walls 211B and 211D.

It is described above how the sections of the passage 220 are generally cylindrical, i.e. with circular cross sections. The corresponding sections of the connecting portion 300 are cooperatively cylindrical with circular cross sections. However, it will be appreciated that any other cross-sectional shapes can be used. For example, the passage sections 221A-221C may have any cross sectional shape, and the sections 311A-311C may have a cooperating cross sectional shape, provided that it enables the electrical contacts and/or seals to function as described herein.

In the illustrated examples, the active contacts 331/231 and the return contacts 341/241 are located along a same line that runs parallel to the axis 390/290. It will be appreciated that the active contacts 331/231 and the return contacts 241/341 may be located along different lines (e.g. at different circumferential positions around the axis 390).

In the illustrated examples, the return contact 341 is located directly on the conductive surface of the elongate shaft 31. However, in some examples, the return contact 341 may also be located on a surface of the connection hub 310. For example, the connection hub 310 may include an additional body section that is received in the passage section 221C and therefore surrounds the proximal portion 31a of the elongate shaft 31. The return contact 341 may be on a surface of said additional body section. The return contact 341 may be connected to the elongate shaft 31 via a wire.

In the illustrated examples, the active and return contacts 331/341 of the connection hub 310) are leaf spring contacts. However, it will be appreciated that the active and return contacts 331/341 may be any other type of electrical contact. For example, any other type of resiliently deformable or spring electrical contact may be used.

It is described above that the sections or segments 221A, 221B, 221C, 221D, 221E are coaxial. However, it will be appreciated that in some examples, one or more of the sections or segments 221A, 221B, 221C, 221D, 221E may be not be coaxial with one another (e.g. one or more of the sections may have offset but parallel axes to one another).

It is described above that the body 309 is coaxial with the elongate shaft 31, e.g. about the axis 390. However it will be appreciated that in some examples, the body 309 may not be coaxial with the elongate shaft 31 (e.g. the body 309 may have an offset, but parallel axis to the elongate shaft 31).

It is described above that the body 309 includes a series of coaxial sections 311A, 311B, 311C. However, it will be appreciated that in some examples, one or more of the sections 311A, 311B, 311C may not be coaxial with one another (e.g. one or more of the sections may have offset but parallel axes to one another).

In the illustrated examples, the sealing rings 321/323 and the grooves 313A/313C are coaxial with the elongate shaft 31. However, it will be appreciated that in some examples, one or more of the sealing rings and/or grooves may not be coaxial with the elongate shaft 31 (e.g. one or more of the sealing rings and/or grooves may have an offset but parallel axis to the elongate shaft or to one another).

It is described above that the connection portion 300 is coaxially coupled with or received in the socket 200. However it will be appreciated that in some examples, the connection portion 300 may be non-coaxially coupled or received in the socket 200.

It will be appreciated that the wire(s) described above may be any other suitable type of electrical conductor.

Reprocessing

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

Various modifications whether by way of addition, deletion, or substitution of features may be made to above described embodiment to provide further embodiments, any and all of which are intended to be encompassed by the appended claims.

There follows a list of numbered features defining particular embodiments of the present disclosure. Where a numbered feature refers to one or more earlier numbered features then those features should be considered together in combination.
1. A surgical instrument for releasably coupling to a handpiece of a surgical device, the surgical instrument comprising:
   an elongate shaft having a distal end and a proximal end, wherein the elongate shaft defines a longitudinal axis in a direction of elongation of the elongate shaft;
   an end effector at the distal end of the elongate shaft, wherein the end effector is for treating tissue; and
   a connecting portion at the proximal end of the elongate shaft, the connecting portion configured to be inserted into a socket of the handpiece and receive an electrical power signal from the handpiece, the connecting portion comprising:
      a first electrical contact configured to align with a corresponding first electrical contact in the socket of the handpiece when the connecting portion is in the socket, and
      a second electrical contact configured to align with a corresponding second electrical contact in the socket of the handpiece when the connecting portion is in the socket,
      wherein the first electrical contact and the second electrical contact are located at different longitudinal positions such that the first electrical contact is more distal than the second electrical contact.
2. The surgical instrument of feature 1, wherein the connecting portion further comprises a first sealing element in between the first electrical contact and the second electrical contact, the first sealing element configured to electrically isolate the first electrical contact from the second electrical contact when the connecting portion is in the socket.
3. The surgical instrument of feature 1 or feature 2, the connecting portion further comprises a second sealing element distal to the first electrical contact, the second sealing element configured to isolate the first electrical contact from an opening of the socket when the connecting portion is in the socket.
4. The surgical instrument of any preceding feature, wherein the connecting portion comprises a connection hub having a body, wherein the first electrical contact is on a surface of the body, optionally wherein the body is substantially coaxial with the elongate shaft.
5. The surgical instrument of feature 5, wherein the elongate shaft extends through the body such that a proximal portion of the elongate shaft protrudes from a proximal side of the body, wherein the second electrical contact is on a surface of the proximal portion of the elongate shaft.
6. The surgical instrument of any preceding feature, wherein the surgical instrument further comprises a first current path configured to conduct electrical current between the first electrical contact and the end effector, and a second current path configured to conduct electrical current between the second electrical contact and the end effector.
7. The surgical instrument of feature 6, wherein the first current path comprises an electrical conductor, and/or wherein at least the surface of the elongate shaft is electrically conductive so as to provide the second current path.
8. The surgical instrument of any of features 4 to 7, wherein the first sealing element comprises a first sealing ring around the surface of the body, optionally wherein the first sealing ring is coaxial with the elongate shaft.
9. The surgical instrument of any of features 4 to 8, wherein the second sealing element comprises a second sealing ring around the surface of the body, optionally wherein the second sealing ring is coaxial with the elongate shaft.
10. The surgical instrument of any of features 8 or 9, wherein the surface of the body comprises a first groove and the first sealing ring is positioned in the first groove, optionally wherein the first groove is coaxial with the elongate shaft; and/or
   wherein the surface of the body comprises a second groove and the second sealing ring is positioned in the second groove, optionally wherein the second groove is coaxial with the elongate shaft.
11. The surgical instrument of any of features 4 to 10, wherein the body of the connection hub comprises a plurality of sections, each section having a substantially circular cross-section, wherein the first electrical contact is on a surface of a first section of the plurality of sections, and the first sealing ring is around a surface of a second section of the plurality of sections, wherein the radius of the first section is greater than the radius of the second section, optionally wherein the plurality of sections are coaxial.
12. The surgical instrument of any of features 4 to 11, wherein the second sealing ring is around a surface of a third section of the plurality of sections, wherein the radius of the third section is greater than the radius of the first section.
13. The surgical instrument of any preceding feature, wherein the first electrical contact and the second electrical contact are resiliently deformable, optionally wherein the first electrical contact ad the second electrical contact are resiliently deformable in a direction towards the longitudinal axis.
14. The surgical instrument of any preceding feature, wherein the instrument further comprises a rotatable shaft arranged concentrically within the elongate shaft, the rotatable shaft comprising a blade for cutting tissue at a distal end thereof.
15. The surgical instrument of any preceding feature, wherein the instrument further comprises a locking mechanism configured to releasably secure the connecting portion within the socket of the handpiece.
16. The surgical instrument of any preceding feature, wherein the surgical instrument is an electrosurgical instrument and/or wherein the electrical power signal is an electrical RF power signal.
17. A handpiece for releasably coupling with a surgical instrument, the handpiece comprising:
   a socket for receiving a connecting portion of the surgical instrument to provide an electrical power signal to the surgical instrument, the socket comprising:
      a distal opening that provides access to a passage defined by an inner wall of the socket, the passage having a longitudinal axis;
      a first electrical contact located in the passage and configured to align with a corresponding first electrical contact of the connecting portion of the surgical instrument when the connecting portion is inserted in the socket, and
      a second electrical contact located in the passage and configured to align with a corresponding second electrical contact of the connecting portion of the surgical instrument when the connecting portion is inserted in the socket,
      wherein the first electrical contact and the second electrical contact are located at different longitudinal positions such that the first electrical contact is more distal than the second electrical contact.
18. The handpiece of feature 17, wherein the passage is formed of a series of passage segments, each passage segment being defined by a respective section of the inner wall, wherein the first electrical contact is located in a first passage segment of the passage segments, and the second electrical contact is located in a second passage segment of the passage segment, optionally wherein the series of passage segments are coaxial.
19. The handpiece of feature 18, wherein the first electrical contact is embedded in the section of the inner wall that defines the first passage segment, and the second electrical contact is embedded in the second of the inner wall that defines the second passage segment.
20. The handpiece of any of features 18 to 19, where the passage further comprises a third passage segment in between the first and the second passage segments, wherein the third passage segment is for receiving a first sealing element of the connecting portion of the surgical instrument to electrically isolate the first electrical contact from the second electrical contact when the connecting portion is inserted into the socket.
21. The handpiece of any of features 18 to 20, wherein the passage further comprises a fourth passage segment distal to the first passage segment, wherein the fourth passage segment is for receiving a second sealing element of the connecting portion of the surgical instrument to isolate the first electrical contact from the opening.
22. The handpiece of any of features 18 to 21, wherein the passage further comprises a fifth passage segment proximal to the second passage segment, wherein the fifth passage segment is for receiving at least part of a rotatable shaft of the surgical instrument.
23. The handpiece of any of features 18 to 22, wherein the passage segments have a substantially circular cross section.
24. The handpiece of feature 23, wherein each passage segment has a radius such that the radius of the passage reduces in the distal-proximal direction.
25. A surgical device comprising:
   the surgical instrument defined in any of features 1 to 16; and
   the handpiece defined in any of features 17 to 24,
   wherein the connecting portion of the surgical instrument is received in the socket of the handpiece.
26. A surgical device comprising:
   a handpiece having a socket comprising:
      a distal opening that provides access to a passage defined by an inner wall of the socket, the passage having a longitudinal axis, and a first electrical contact and a second electrical contacted each located in the passage, wherein the first electrical contact and the second electrical contact are displaced along the longitudinal axis of the passage such that the first electrical contact is more distal than the second electrical contact; and
   a surgical instrument comprising:
      an elongate shaft having a distal end and a proximal end, wherein the elongate shaft defines a longitudinal axis in a direction of elongation of the elongate shaft,
      an end effector at the distal end of the elongate shaft, wherein the end effector is for treating tissue, and
      a connecting portion comprising a third electrical contact and a fourth electrical contact, wherein the third electrical contact and the fourth electrical contact are displaced along the longitudinal axis of the elongate shaft such that the third electrical contact is more distal than the fourth electrical contact,
   wherein the surgical instrument is coupled to the handpiece by the connecting portion being received in the passage, such that the first electrical contact is aligned with the third electrical contact and the second electrical contact is aligned with the fourth electrical contact to provide an electrical power signal from the handpiece to the surgical instrument.
27. The surgical device of feature 26, wherein the connecting portion further comprises at least a first sealing element in between the third electrical contact and the fourth electrical contact, the first sealing element engages the inner wall of the socket between the first electrical contact and the fourth electrical contact to electrically isolate the first electrical contact and the third electrical contact from the second electrical contact and the fourth electrical contact.
28. A surgical system, comprising:
   an RF electrosurgical generator;
   a suction source; and
   a surgical device according to any of features 25 to 27, the arrangement being such that in use the generator supplies on demand an RF electrical power signal to the handpiece, the RF electrical power signal being an RF cutting, coagulation or ablation signal.
29. A method of reconditioning a surgical instrument according to any of features 1 to 16, comprising:
   a) receiving a used surgical instrument according to any of features 1 to 16;
   b) disassembling the surgical instrument;
   c) cleaning and reconditioning the surgical instrument, the reconditioning including the replacement of any worn or single use parts;
   d) reassembling the cleaned and reconditioned surgical instrument;
   e) packing the re-assembled instrument in surgical instrument packaging; and
   f) sterilising the re-assembled and packed instrument.

1. A surgical instrument for releasably coupling to a handpiece of a surgical device, the surgical instrument comprising:
an elongate shaft having a distal end and a proximal end, wherein the elongate shaft defines a longitudinal axis in a direction of elongation of the elongate shaft;
an end effector at the distal end of the elongate shaft, wherein the end effector is for treating tissue; and
a connecting portion at the proximal end of the elongate shaft, the connecting portion configured to be inserted into a socket of the handpiece and receive an electrical power signal from the handpiece, the connecting portion comprising:
a first electrical contact configured to align with a corresponding first electrical contact in the socket of the handpiece when the connecting portion is in the socket, and
a second electrical contact configured to align with a corresponding second electrical contact in the socket of the handpiece when the connecting portion is in the socket,
wherein the first electrical contact and the second electrical contact are located at different longitudinal positions such that the first electrical contact is more distal than the second electrical contact.

2. The surgical instrument of claim 1, wherein the connecting portion further comprises a first sealing element in between the first electrical contact and the second electrical contact, the first sealing element configured to electrically isolate the first electrical contact from the second electrical contact when the connecting portion is in the socket.

3. The surgical instrument of claim 1, the connecting portion further comprises a second sealing element distal to the first electrical contact, the second sealing element configured to isolate the first electrical contact from an opening of the socket when the connecting portion is in the socket.

4. The surgical instrument of claim 1, wherein the connecting portion comprises a connection hub having a body, wherein the first electrical contact is on a surface of the body, optionally wherein the body is substantially coaxial with the elongate shaft.

5. The surgical instrument of claim 1, wherein the elongate shaft extends through the body such that a proximal portion of the elongate shaft protrudes from a proximal side of the body, wherein the second electrical contact is on a surface of the proximal portion of the elongate shaft.

6. The surgical instrument of claim 1, wherein the surgical instrument further comprises a first current path configured to conduct electrical current between the first electrical contact and the end effector, and a second current path configured to conduct electrical current between the second electrical contact and the end effector.

7. The surgical instrument of claim 4, wherein the body of the connection hub comprises a plurality of sections, each section having a substantially circular cross-section, wherein a first electrical contact is on a surface of a first section of the plurality of sections, and a first sealing ring is around a surface of a second section of the plurality of sections, wherein the radius of the first section is greater than the radius of the second section.

8. The surgical instrument of claim 7, wherein a second sealing ring is around a surface of a third section of the plurality of sections, wherein a radius of the third section is greater than the radius of the first section.

9. The surgical instrument of claim 1, wherein the first electrical contact and the second electrical contact are resiliently deformable, wherein the first electrical contact and the second electrical contact are resiliently deformable in a direction towards the longitudinal axis.

10. The surgical instrument of claim 1, wherein the instrument further comprises a rotatable shaft arranged concentrically within the elongate shaft, the rotatable shaft comprising a blade for cutting tissue at a distal end thereof.

11. The surgical instrument of claim 1, wherein the instrument further comprises a locking mechanism configured to releasably secure the connecting portion within the socket of the handpiece.

12. The surgical instrument of claim 1, wherein the surgical instrument is an electrosurgical instrument and/or wherein the electrical power signal is an electrical RF power signal.

13. A handpiece for releasably coupling with a surgical instrument, the handpiece comprising:
a socket for receiving a connecting portion of the surgical instrument to provide an electrical power signal to the surgical instrument, the socket comprising:
a distal opening that provides access to a passage defined by an inner wall of the socket, the passage having a longitudinal axis;
a first electrical contact located in the passage and configured to align with a corresponding first electrical contact of the connecting portion of the surgical instrument when the connecting portion is inserted in the socket, and
a second electrical contact located in the passage and configured to align with a corresponding second electrical contact of the connecting portion of the surgical instrument when the connecting portion is inserted in the socket,
wherein the first electrical contact and the second electrical contact are located at different longitudinal positions such that the first electrical contact is more distal than the second electrical contact.

14. The handpiece of claim 13, wherein the passage is formed of a series of passage segments, each passage segment being defined by a respective section of the inner wall, wherein the first electrical contact is located in a first passage segment of the passage segments, and the second electrical contact is located in a second passage segment of the passage segment, optionally wherein the series of passage segments are coaxial.

15. The handpiece of claim 14, wherein the first electrical contact is embedded in the section of the inner wall that defines the first passage segment, and the second electrical contact is embedded in the second of the inner wall that defines the second passage segment.

16. The handpiece of claim 14, where the passage further comprises a third passage segment in between the first and the second passage segments, wherein the third passage segment is for receiving a first sealing element of the connecting portion of the surgical instrument to electrically isolate the first electrical contact from the second electrical contact when the connecting portion is inserted into the socket.

17. The handpiece of claim 14, wherein the passage further comprises a fourth passage segment distal to the first passage segment, wherein the fourth passage segment is for receiving a second sealing element of the connecting portion of the surgical instrument to isolate the first electrical contact from the opening, and wherein the passage further comprises a fifth passage segment proximal to the second passage segment, wherein the fifth passage segment is for receiving at least part of a rotatable shaft of the surgical instrument.

18. The handpiece of claim 14, wherein the passage segments have a substantially circular cross section, wherein each passage segment has a radius such that the radius of the passage reduces in the distal-proximal direction.

19. A surgical device comprising:
a handpiece having a socket comprising:
a distal opening that provides access to a passage defined by an inner wall of the socket, the passage having a longitudinal axis, and a first electrical contact and a second electrical contacted each located in the passage, wherein the first electrical contact and the second electrical contact are displaced along the longitudinal axis of the passage such that the first electrical contact is more distal than the second electrical contact; and
a surgical instrument comprising:
an elongate shaft having a distal end and a proximal end, wherein the elongate shaft defines a longitudinal axis in a direction of elongation of the elongate shaft,
an end effector at the distal end of the elongate shaft, wherein the end effector is for treating tissue, and
a connecting portion comprising a third electrical contact and a fourth electrical contact, wherein the third electrical contact and the fourth electrical contact are displaced along the longitudinal axis of the elongate shaft such that the third electrical contact is more distal than the fourth electrical contact,
wherein the surgical instrument is coupled to the handpiece by the connecting portion being received in the passage, such that the first electrical contact is aligned with the third electrical contact and the second electrical contact is aligned with the fourth electrical contact to provide an electrical power signal from the handpiece to the surgical instrument.

20. The surgical device of claim 19, wherein the connecting portion further comprises at least a first sealing element in between the third electrical contact and the fourth electrical contact, the first sealing element engages the inner wall of the socket between the first electrical contact and the fourth electrical contact to electrically isolate the first electrical contact and the third electrical contact from the second electrical contact and the fourth electrical contact.
